(19) United States
(12) Patent Application Publication
(10) Pub. No.: US 2021/0193345 A1
(43) Pub. Date: Jun. 24, 2021

(54) X-RAY REFLECTIVE LENS ARRANGEMENT

(71) Applicant: CONVERGENT R.N.R LTD., Tirat Carmel (IL)

(72) Inventors: Aharon BAR-DAVID, Nesher (IL); Zeev HAREL, Kfar Saba (IL); Zeev BURSHTEIN, Ness-Ziona (IL)

(21) Appl. No.: 17/196,340

(22) Filed: Mar. 9, 2021

Related U.S. Application Data
(63) Continuation-in-part of application No. 16/543,751, filed on Aug. 19, 2019.
Application No. 16/543,751 is a reissue of application No. 14/430,683, filed on Mar. 24, 2015, now Pat. No. 9,953,735.
(60) Provisional application No. 61/704,588, filed on Sep. 24, 2012.

(51) Int. Cl. G21K 5/04; A61B 6/00; G21K 1/06; A61N 5/10

(52) U.S. Cl. CPC G21K 5/04; A61B 6/584; G21K 2201/064; A61N 5/1042; G21K 1/062

(57) ABSTRACT

An X-ray lens arrangement for forming a radiation pattern as a focal track is disclosed. The pattern comprises at least one 3-dimensional focal track of radiation. The aforesaid lens arrangement has a main axis passing through intensity weighted centroids of the Xray source and the pattern. The lens arrangement includes at least one reflecting surface of continuously varying Rowland arcs. Each point belonging to the focal track is linked to each elemental point composing an emitting surface of said source by a corresponding Rowland arc.

### CROSS-REFERENCE TO RELATED APPLICATIONS

This application is a Continuation-in-part of U.S. patent application Ser. No. 16/543,751 filed Aug. 19, 2019, which is a reissue of U.S. Pat. No. 9,953,735 issued on Apr. 24, 2018 and having a U.S. application Ser. No. 14/430,683 and filing date of Mar. 24, 2015, which is a U.S. National Phase of PCT Patent Application No. PCT/IL2013/050739 having International filing date of Sep. 1, 2013, which claims the benefit of priority of U.S. Provisional Application No. 61/704,588 filed Sep. 24, 2012, the contents of which are all incorporated herein by reference in their entirety.

### FIELD OF THE INVENTION

The present invention relates to an X-ray lens and, more specifically, to an X-ray lens arrangement configured for focusing a radiation from an X-ray source into a customizable radiation pattern in a volume of radiotherapy treatment.

### BACKGROUND OF THE INVENTION

According to conventional radiation therapy, a radiation beam is directed towards a tumor located within a patient's body. The radiation beam delivers a predetermined dose of therapeutic radiation to the tumor according to an established therapy plan. The delivered radiation kills tumor cells by causing ionizations within the cells. In this regard, radiation therapy systems are designed to maximize radiation delivered to the tumor while minimizing radiation delivered to healthy tissue.

U.S. Pat. No. 6,389,100 discloses a modular X-ray lens system for use in directing X-rays comprising a radiation source which generates X-rays and a lens system which directs the X-ray beam. The X-ray lens system is configured to focus X-rays to a focal point and vary the intensity of said focal point.

U.S. Pat. No. 7,068,754 discloses an X-ray apparatus including a ring anode to emit radiation, and a conical monochromator to monochromatize the emitted radiation. An outer diameter of the ring anode is greater than an outer diameter of a base of the monochromator.

### SUMMARY OF THE INVENTION

It is hence one object of the invention to disclose an X-ray reflective lens arrangement for forming a radiation pattern in a focal region. The aforesaid lens arrangement is longitudinally arranged for Bragg X-ray diffraction of said X-rays.

It is a further core purpose of the invention to provide the arrangement comprises at least one continuous reflecting surface defined by arcs locally belonging to Rowland circles of continuously varying radii. At least one reflecting surface is configured for reflecting said X-rays such that any elemental point composing an emitting surface of the X-ray source is imaged into a corresponding point belonging to a focal track formed by reflected X-rays within the Rowland circles of the continuously varying radii.

Another object of this disclosure is to disclose at least one continuous reflecting surface formed by a flexible crystal arrangement. The flexible crystal arrangement is movable by an actuator which enables dynamically varying local Rowland radii of said continuous reflective surface and controlling a shape of said focal track.

A further object of this disclosure is to disclose the actuator comprising at least one piezoelectric drive.

### BRIEF DESCRIPTION OF THE DRAWINGS

In order to understand the invention and to see how it may be implemented in practice, a plurality of embodiments is now be described, by way of non-limiting example only, with reference to the accompanying drawings, in which

FIG. 1 is a schematic partial longitudinal cross-sectional view of a crystal element with schematic reflection planes of an X-ray lens;

FIG. 2 is a two-dimensional diagram of the Johansson scheme;

FIG. 3 is a schematic presentation of the elemental reflective lens;

FIG. 4 is a general schematic view of the lens arrangement;

FIGS. 5 and 6 are schematic views of the exemplary embodiments of the lens arrangement;

FIG. 7 is a schematic view of an exemplary embodiment of an open lens arrangement;

FIG. 8 a schematic view of the lens arrangement comprising the plurality of reflective tile surfaces; and

FIG. 9 is an overall view of a single X-ray tile reflector having a support surface dynamically controlled by piezoelectric tiles.

### DETAILED DESCRIPTION OF THE INVENTION

The following description is provided, alongside all chapters of the present invention, so as to enable any person skilled in the art to make use of the aforesaid invention, and sets forth the best modes contemplated by the inventor of carrying out this invention. Various modifications, however, are adapted to remain apparent to those skilled in the art, since the generic principles of the present invention have been defined specifically to provide an X-ray reflective lens arrangement for forming an intensity pattern in a focal region and methods of using the same.

The term “elemental” hereinafter refers to infinitely small portion of a physical entity.

The term “focal track” hereinafter refers to an ordered ensemble of elemental focal points created by a reflecting surface of an X-ray lens.

The term “intensity weighted centroid of the X-ray source” hereinafter refers to a point defined by a vector r̅_{sc}

$$\vec{{r}_{sc}} = \frac{{\int }_{complete source shape} {I}_{source} ( x , y , z ) \vec{r} dV}{{\int }_{complete source shape} {I}_{source} ( x , y , z ) dV} = \frac{{\int }_{complete source shape} {I}_{source} ( x , y , z ) \vec{r} dV}{{I}_{source \_ total}} .$$

The term “intensity weighted centroid of the focal pattern” hereinafter refers to a point defined by a vector

$$\vec{{r}_{fc}} = \frac{{\int }_{complete focal shape} {I}_{focus} ( x , y , z ) \vec{r} dV}{{\int }_{complete focal shape} {I}_{focus} ( x , y , z ) dV} = \frac{{\int }_{complete focal shape} {I}_{focus} ( x , y , z ) \vec{r} dV}{{I}_{focus \_ total}} .$$

I_{focus}(x,y,z) is a spatial distribution of radiation intensity in the focal region, and I_{source}(x,y,z,) is the spatial distribution of source intensity at the source space. It should be appreciated that the radiation pattern has a three-dimensional shape.

Referring to the medical use of the X-ray system for tumor treatment, the known therapeutic devices comprising focusing elements are characterized by concentration of X-ray radiation into a sharp focal spot. It should be emphasized that uniform X-ray exposure of a target volume is a desirable condition of successful therapy or surgery because the optimal effect is achieved when all target tissue is exposed to a uniform dose.

Thus, there is a long-felt and unmet need to provide a therapeutic device for X-ray treatment of tumors adapted for forming substantially uniform X-ray intensity within the target volume.

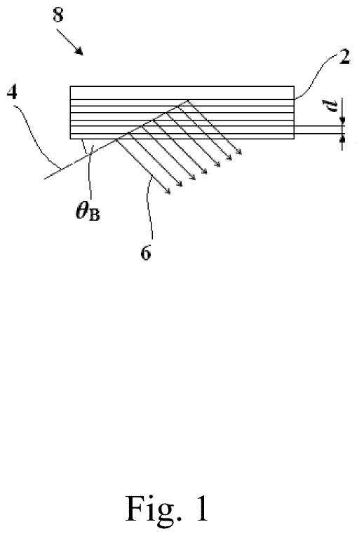

Reference is now made to FIG. 1, illustrating a simple Bragg reflector utilizing the principles of Bragg reflection. X-ray radiation 4 of wavelength λ is incident on a crystal having lattice planes 2 of plane spacing d. Narrow band or generally monochromatic radiation 6 is then reflected according to Bragg's Law. Bragg structures only reflect radiation when Bragg's equation is satisfied:

nλ=2d sin θ_{B}, (1)

where n is the reflection order, λ is the incident radiation wavelength, d is the lattice plane spacing, and θ_{B} is the Bragg angle.

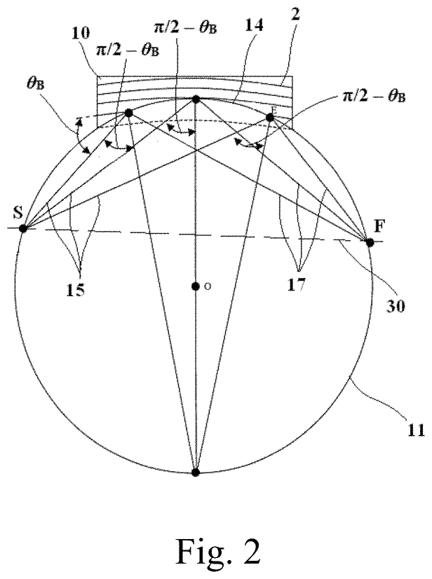

Reference is now made to FIG. 2, presenting a two-dimensional longitudinal cut of the Johansson scheme. A Johansson bent and machined crystal 10 is used to reflect and focus X-rays. The Johansson bent and machined crystal 10 reflects X-rays according to Bragg's law. The Johansson crystal 10 is made by bending and grinding a crystal into a barrel shaped surface with a longitudinal bending radius 2R, and then the reflection surface 14 is machined to a cylindrical surface with longitudinal radius R. In a special symmetrical case, the angles of incidence of rays 15 generated by the X-ray source S and angles of reflection of rays 17 converging into the point F, are equal.

The transversal curvature radius of the machined surface at a midpoint between the source and the focal point s r is given by

rₛ=L tan θ_{B}, (2)

L is half of the distance from the source to the focal point.

The Rowland radius R is given by the following expression

$$R = \frac{{r}_{s}}{2 {sin}^{2} {θ}_{B}} \qquad ( 3 )$$

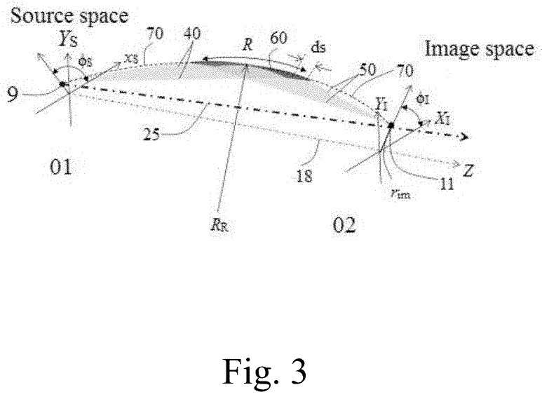

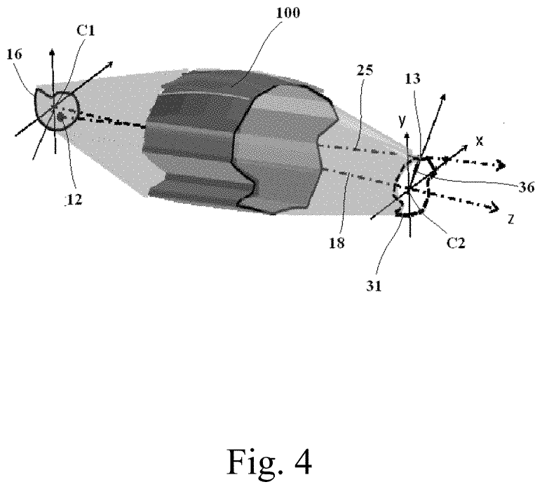

Reference is now made to FIG. 3, elucidating a subject matter of the current invention. An elemental point 11 is a part of the image of an elemental X-ray source point 9 in source space X_{S}Y_{S} formed by an elemental portion 60 of reflective lens which lies in a Rowland arc 70 subtended by a chord 25. In other words, elemental portion 60 is locally defined by radius R_{R} which continuously varies over reflective surface of lens arrangement 100 (see FIG. 4). The reflecting surface is configured for reflecting said X-rays such that any elemental point 9 composing emitting surface of said X-ray source is imaged into a corresponding point belonging to a focal track formed by reflected X-rays within said Rowland circles of continuously varying radii Rowland arc.

Lines 40 and 50 refer to rays emitted by the X-ray source elemental point 9 and reflected from the lens portion 60, respectively. An axis 18 is a main axis of the entire lens. The chord 25 is the optical axis of the narrow elemental reflective lens portion 60. The aforesaid point 11 is at location rᵢₘ on the X_{I}Y_{I} plane of the image space.

The elemental point source 9 makes an angle ϕ_{S} relative to the X_{S} axis in source space.

The elemental point 11 makes an angle ϕ_{I} relative to the X_{I} axis in image space, wherein ϕ_{S} and ϕ_{I} are generally not the same, thus, in general, the image point 11 can be rotated relative to the source point 9.

Reference is now made to FIG. 4, presenting a lens arrangement 100 continuously defined by an ensemble of elemental arcs 60 being rotated around the main axis 18. On the basis of continuously variable Rowland radii R_{R} of elemental arcs 60 (FIG. 3) forming the reflective surface, the lens arrangement is designed to provide a customizable reflective surface which enables focusing X-rays emitted by the X-ray source into any arbitrary radiation pattern. The lens arrangement 100 focuses radiation emitted by the X-ray source 16 into a curved radiation pattern 31. It should be emphasized that the curved pattern of radiation pattern 31 is an ensemble of elemental points 13 created by the plurality of elemental arcs 60 integrally forming the reflective surface 100. One ray is shown from the single point 12 on the source 16 to a point 13 on the focal track 31.

The main axis 18 is defined by two points which are: (1) the intensity weighted centroid C1 of the X-ray source, and (2) a centroid C2 of the linear radiation pattern 30. The centroids are intensity weighted average points of the source and the radiation patter 31.

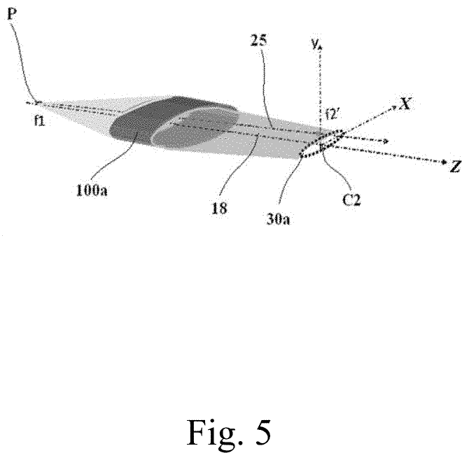

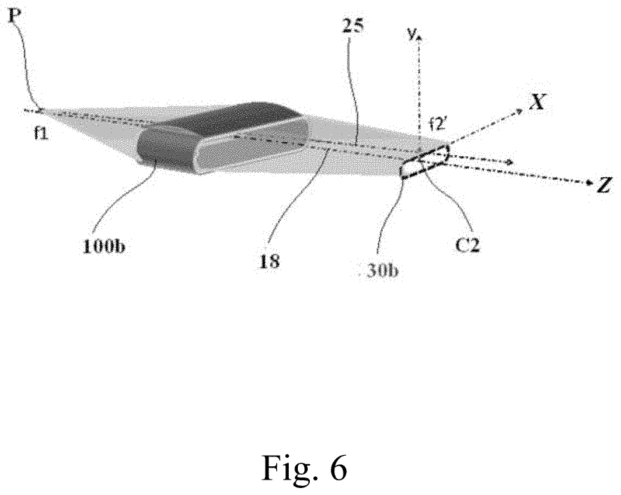

Reference is now made to FIGS. 5 and 6, presenting exemplary embodiments of the current invention. Specifically, a lens arrangement 100a is configured to provide an elliptic radiation pattern 30a while a lens arrangement 100b focuses radiation from the X-ray source into an orthogon 30b with rounded angles. The designation P refers to a point source.

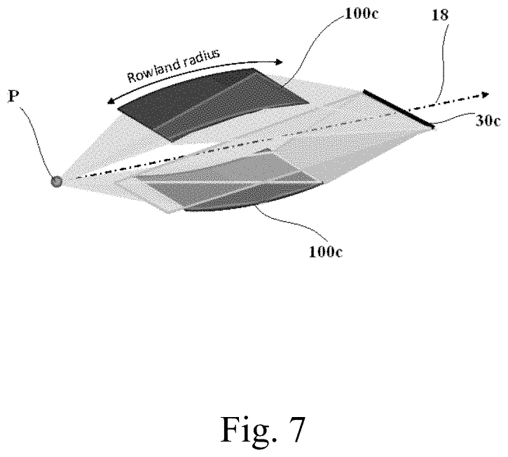

Reference is now made to FIG. 7, presenting an alternative embodiment of the current invention. A lens arrangement 100c is portioned into two parts, which are configured to provide the X-ray radiation into same curved radiation pattern 30c.

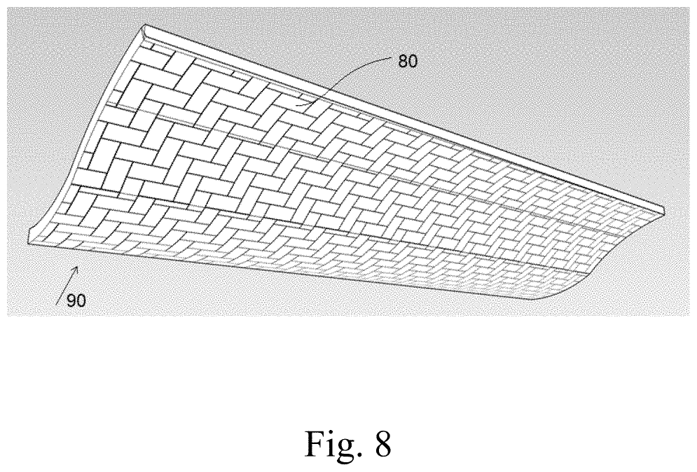

Reference is now made to FIG. 8 showing an overall view of an exemplary embodiment of dynamically controlled reflecting surface 90 which allows changing a local curvature (Rowland radius) in real time and, consequently, the resultant focal pattern. According to one embodiment of the present invention, dynamically controlled reflecting surface 90 is embodied as a plurality of tiles 80 angularly movable by plurality of actuators in an individual manner.

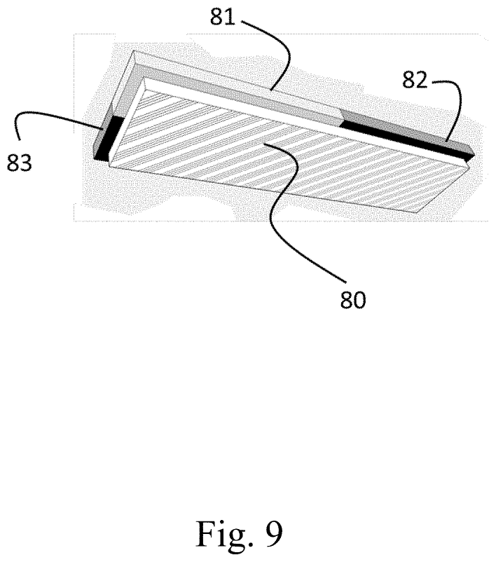

Reference is now made to FIG. 9 presenting a single reflective tile mounted on a dynamically controlled support surface which is a mean of controlling the orientation of a single tile, for example the figure shows 4 piezo electric surface 81, 82 and 83 (the 4^{th} is hidden so it's not shown), which by electrical voltage can be made thicker of thinner changing the orientation of the reflecting tile that is mounted on it. Thus, the movement of the piezo surface in the direction perpendicular to the tile plane can be actuated by electric voltage and is also in the scope of the present invention.

An additional benefit of the current invention is in the use of single crystals exhibiting some degree of mosaicity. The focal tracks thus created by the present invention are characterized by three-dimensional broadening which serves the purpose of allowing for homogeneity of the created radiation pattern within the target volume.

Special benefits can be made in cases where the body has to be irradiated from the front, e.g. after breast mastectomy. The existing technology provides irradiation of the entire depth of the body over relatively large area. The current invention provides a high convergence angle. Thus, utilizing the high convergence angle yields a large attenuation after the target volume, spearing healthy tissues.

1. An X-ray reflective lens arrangement for forming a radiation pattern in a focal region; said lens arrangement being longitudinally arranged for Bragg X-ray diffraction of said X-rays; wherein said arrangement comprises at least one continuous reflecting surface defined by arcs locally belonging to Rowland circles of continuously varying radii; said at least one reflecting surface is configured for reflecting said X-rays such that any elemental point composing an emitting surface of said X-ray source is imaged into a corresponding point belonging to a focal track formed by reflected X-rays within said Rowland circles of continuously varying radii Rowland arc.

2. The lens arrangement according to claim 1, wherein said at least one continuous reflecting surface is formed by a flexible crystal arrangement; said flexible crystal arrangement is movable by an actuator which enables dynamically varying local Rowland radii of said continuous reflective surface and controlling a shape of said focal track.

3. The lens arrangement according to claim 1, wherein said actuator comprises at least one piezoelectric drive.
